(19) United States
(12) Patent Application Publication
(10) Pub. No.: US 2005/0014255 A1
(43) Pub. Date: Jan. 20, 2005

(54) Stem cells for clinical and commercial uses

(75) Inventors: Liping Tang, Arlington, TX (US); Robert H. Hansen, Lewisville, TX (US)

Correspondence Address: KENNETH R. GLASER;THOMAS C. WRIGHT, GARDERE WYNNE SEWELL LLP, 1601 ELM STREET, SUITE 3000, DALLS, TX, 75201, US

(73) Assignee: BOARD OF REGENTS, THE UNIVERSITY OF TEXAS SYSTEM, Austin, TX (US)

(21) Appl. No.: 10/857,443

(22) Filed: May 27, 2004

Related U.S. Application Data
(60) Provisional application No. 60/474,020, filed on May 29, 2003.

(51) Int. Cl. A61K 45/00; C12N 5/08; A61M 31/00

(52) U.S. Cl. 435/366; 424/93.7; 604/500

(57) ABSTRACT

Methods for accumulating, recruiting and retrieving stem cells from a body cavity space of an adult mammal are disclosed, in addition to compositions and cells in accordance with the present invention. In accordance with another aspect of the present invention, a method of obtaining a new source of stem cells is provided by implanting a foreign object in a body cavity space of a mammal and collecting those stem cells resulting from implantation. Further in accordance with the present invention, a method for providing to a subject in need thereof one or more stem cells of the present invention is identified by retrieving stem cells for a body cavity space of a mammal after implanting a foreign object, manipulating the stem cells and introducing the stem cells into the subject in need thereof.

This application claims priority to U.S. Provisional Patent Application Ser. No. 60/474,020, filed May 29, 2003.

### STATEMENT REGARDING FEDERALLY SPONSORED RESEARCH OR DEVELOPMENT

The present application was supported in part by the National Institutes of Health grant number EB-00287. The government may have certain rights in the invention.

### BACKGROUND OF THE INVENTION

The present invention relates to the general field of cell physiology, and more particularly to compositions and methods of developing and producing clinically viable stem cells for medical applications, such as diagnosis, screening, testing, therapy, and rehabilitation, as well as cells for use in commercial applications, such as screening, testing, and bioengineering.

Stem cell research provides a new panacea for patients with cancer, spinal cord injuries, stroke, degenerative diseases, and other conditions because of their plasticity and potential use to replace diseased, injured or aged tissues and organs. It has been suggested that by using stem cell transplants instead of drugs, biologics, and other current therapies, stem cells can offer new therapies for the prevention and/or treatment of various human disorders and conditions.

For years, scientists have known that stem cells can be derived from an embryo, adult bone marrow and other tissue, or a fetus (e.g., umbilical cord blood). Due to ethical concerns regarding the retrieval of stem cells from embryonic tissue, this particular area of research has encountered significantly less attention of late. Research has instead focused on the isolation, proliferation, and tissue/cell transplantation of stem cells derived from adult and fetal tissue. One limitation to the use of these types of stem cells is that because the cells are capable of triggering transplant rejection, stem cells from the same patient are generally required. Another limitation is that only a small number of adult or fetal stem cells may be retrieved from any one tissue. This has hindered the use of stem cells for widespread or cost-effective clinical treatment. To date, the most abundant source of adult stem cells is from the bone marrow. However, currently less than 500,000 adult stem cells are recovered from the 15 mL of bone marrow fluid (an amount generally retrieved from a human adult). In addition, these stem cells require several weeks or months of cell culturing before producing a decent number of cells for use, such as for transplantation. Furthermore, human liquid marrow cultures often fail to produce significant numbers of either nonadherent hematopoietic precursor cells or clonogenic progenitor cells after 6 to 8 weeks.

Yet another limitation is that only a few types of adult tissues have been reported to contain stem cells. This includes brain, bone marrow, peripheral blood, blood vessels, skeletal muscle, heart, skin and liver. More importantly, stem cells retrieved from adult tissue (also referred to herein as adult stem cells) are often limited in their ability to differentiate. Most often, adult stem cells are only able to differentiate into the cell type of the tissue of origin. As such, most adult stem cells cannot be considered truly pluripotent.

With current techniques, there remain several disadvantages in using adult stem cells. Specifically, adult stem cells are rare in mature tissue and methods for recovery and expansion in culture are generally inefficient, result in a very low yield and are destructive to the tissue—part if not all of the original tissue is destroyed in the process of obtaining stem cells. This makes it difficult to use adult stem cell for replacement or enhancement therapy where large numbers of cells are needed and/or the tissue of origin is essential and cannot be destroyed. Thus, there is a need to improve current stem cell retrieval techniques in order to increase stem cell yield without destroying the tissue of origin. By eliminating the destruction of a tissue, an adult may, after donating stem cells, be the recipient of the stem cells. Such a process will eliminate rejection by the immune system and reduce or eliminate the need for lifetime use of immunosuppressive drugs.

The present invention addresses many of the needs mentioned above as well as other objectives that will be appreciated by those skilled in the art.

### SUMMARY OF THE INVENTION

The present invention solves the problem associated with current stem cell collection and retrieval techniques by providing a new and improved method of obtaining stem cells at a high yield without destroying the tissue of origin as well as methods of use of such stem cells. In addition, the present invention provides populations of stem cells, stem cell lines, and compositions of stem cells for clinical, diagnostic, pharmaceutical, and commercial use.

In accordance with one aspect of the present invention, a method of obtaining a new source of stem cells is provided by introducing a foreign object or an implant into a body cavity space of a mammal and collecting those stem cells resulting from implantation.

Further in accordance with the present invention, a method for providing to a subject in need thereof one or more stem cells of the present invention is identified by retrieving stem cells from a body cavity space of a mammal after implanting a foreign object, manipulating the stem cells and introducing the stem cells into the subject in need thereof. The stem cell may be manipulated by genetics, allowed to contact another composition, directed to specialize into a desired cell type, and/or allowed to contact a three-dimensional scaffold prior to introducing into the subject. In addition, the subject may be the same as the source of the stem cells or different.

Advantages of stem cells retrieved in accordance with the present invention are that they are inexpensive to retrieve, produce a high yield of cells, are capable of differentiation, proliferation, and genetic modification (in vivo and ex vivo), function in a physiologic manner (e.g., conduct, produce, secrete, regulate biologic compounds, etc.), exhibit true pluripotency, and are amenable for clinical, diagnostic and commercial uses, such as cell/tissue transplantation, replacement, implantation, grafting, genetic or diagnostic screenings, product development, and for therapeutic, preventative or other treatments purposes (e.g., for spinal cord injuries, other tissue or organ injuries, burns, cirrhosis, hepatitis, Parkinson's Disease, Alzheimer's Disease, stroke, muscular dystrophy, diabetes, arthritis, osteoporosis, leukemia, sickle cell disease and other anemias, as examples). In addition, the present invention is well suited for use in individuals who may be sensitive to other treatment options such as persons with cancer or those at a late-stage in their disease, an option not available when using most other traditional methods of stem cell retrieval, such as bone marrow harvest which may only be performed in patients considered relatively healthy. Moreover, the present invention is safer than current traditional methods such as bone marrow harvest with no added complications, and may be used repeatedly even on the same patient without any known difficulties or side effects.

The present invention addresses current problems regarding transplant rejection by the immune system and reduce or eliminate the need for use of immunosuppressive drugs with transplantation. The present invention provides an economic clinical treatment option and fulfills the current need of researchers, healthcare providers, and industry for compositions and method of recruiting and retrieving a large number of human stem cells for clinical procedures, including transplantation, gene, protein and cell therapy. The compositions and methods of the present invention serve as unique and powerful tools to supply stem cells, especially to a person in need thereof. Custom designed products of the present invention include compositions for use in medicinal, therapeutic, diagnostic, engineering, and biotechnology applications, as examples.

Those skilled in the art will further appreciate the above-mentioned advantages and superior features of the invention, together with other important aspects thereof upon reading the detailed description that follows in conjunction with the drawings.

### BRIEF DESCRIPTION OF THE SEVERAL VIEWS OF THE DRAWINGS

For a more complete understanding of the features and advantages of the present invention, reference is now made to the detailed description of the invention along with the accompanying FIGURES in which corresponding numerals in the different FIGURES refer to corresponding parts and in which:

FIG. 1 is an example illustrating the pluripotent nature of stem cells of the present invention;

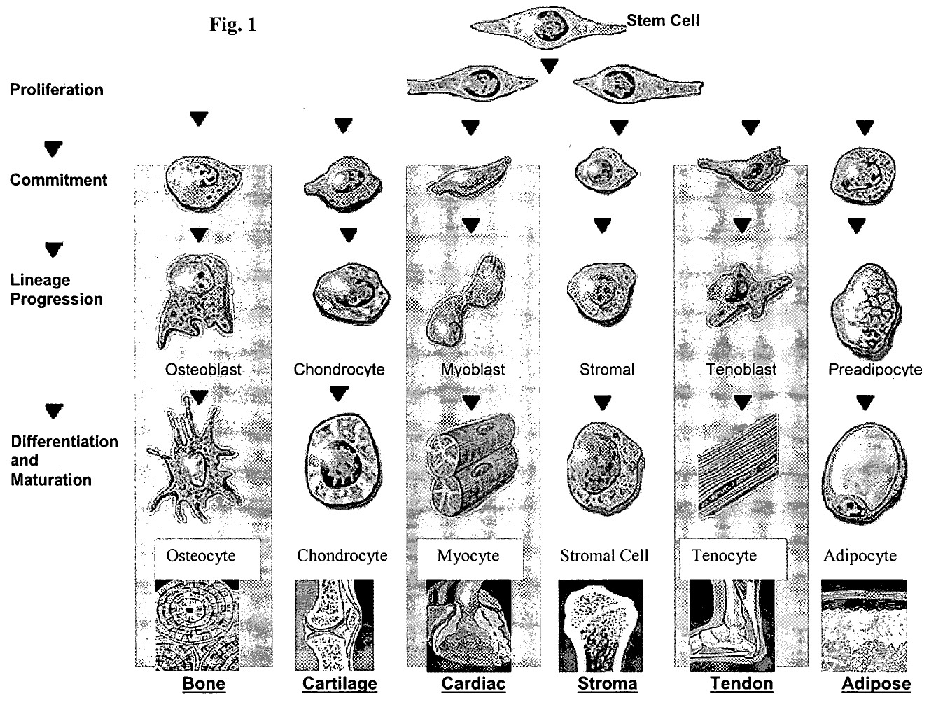

FIG. 2 depicts a light microscopic view of cells of the present invention after retrieval and plating on a substrate;

FIG. 3 depicts the relationship between time and stem cell production in accordance with the present invention, depicting cells exposed to an implant (diamond) and not exposed to an implant (square);

FIG. 4 depicts fibrocyte growth over time in accordance with the present invention, depicting those exposed to an implant with or without lavage fluid (diamond or square, respectively) and not exposed to an implant (crosses or triangles);

FIG. 5 shows two views of cells of the present invention (A) induced to form calcium rich-osteoblast aggregates and (B) control or non-differentiated cells depicting a random distribution;

FIG. 6 depicts a view of cells of the present invention after having been induced to become specialized nerve cells;

FIGS. 7A and 7B depict views of stem cells of the present invention further induced to become specialized nerve cells with typical neuronal morphology;

FIGS. 8A and 8B depict views of stem cells of the present invention induced to become specialized nerve cells by displaying typical neuronal morphology;

FIGS. 9A, 9B and 9C depict views of stem cells of the present invention induced to become specialized nerve cells after staining positive for NFM and FIG. 9D show the stem cells not induced to differentiate and showing no positive NFM staining;

FIG. 10 depict views of stem cells of the present invention induced to become specialized muscle cells with muscle striations (10A and 10B), and linear growth patterns (10C) as well as ability to form myotubes (10D), wherein anchors are striated at the ends of the myotubes after prolonged culturing;

FIG. 11 depicts cell growth as a function of time in accordance with the present invention, depicting exposure to GM-CSF and IL-4 (diamonds) as compared with non growth-inducing signals (squares);

FIG. 12 depicts views of cells of the present invention following the addition of one or more differentiation-inducing signals that promote specialization of the cells into dendritic cells;

FIG. 13 shows several panel views of stem cells of the present invention induced to differentiate into dendritic cells after which immunocytochemistry was performed, wherein arrows in FIGURES A and B show some of the CD11c positive cells, a unique marker for dendritic cells, as compared with control cells not induced to differentiate and displaying no CD11c (no positive staining); and

FIG. 14 depicts images of cells of the present invention following the addition of one or more differentiation-inducing signals that promote specialization of cells into adipocyte cells (FIGS. 14A, 14B, and 14C), wherein differentiated stem cells are oil red o positive (darkened areas) and cells not induced to differentiate into adipocyte cells are not oil red o positive (no darkened areas, FIG. 14D).

### DETAILED DESCRIPTION OF THE INVENTION

Although making and using various embodiments of the present invention are discussed in detail below, it should be appreciated that the present invention provides many inventive concepts that may be embodied in a wide variety of contexts. The specific aspects and embodiments discussed herein are merely illustrative of ways to make and use the invention, and do not limit the scope of the invention.

In general, a stem cell is a pluripotent cell in an unspecialized (e.g., undifferentiated) state that may give rise to one or more unspecialized cells. One critical identifying feature of a stem cell is its ability to exhibit self-renewal or to generate more of itself; therefore, a cell with the capacity for self-maintenance. In addition, as used herein, a stem cell is an unspecialized cell capable of proliferation (replication many times over), self-maintenance, and production of a large number of specialized functional progeny, as well as an ability to regenerate tissue after injury.

One role of stem cells is to replace cells that are lost by natural cell death, ageing, injury or disease. The presence of stem cells in a tissue usually correlates with a high turnover of cells in that tissue, but may also exist in tissue lacking a high turnover rate. Unlike specialized cells that do not have the ability to replicate many times (referred to as “replication” or “proliferation”), adult stem cells should be able to replicate without differentiating for many months. However, current methods retrieve adult stem cells with only a finite ability to grow/proliferate before differentiation is initiated (sometimes automatically) by the cells. Among others, an advantage of the present invention is that it provides for the retrieval of stem cells with an ability to replicate for many months (even greater than a year) while remaining unspecialized and, thus, provides a method of yielding millions of cells for long-term self-renewal.

Another interesting property of stem cells is that they typically generate the same cell type as the tissue from which they are derived. For examples blood-forming adult stem cell from bone marrow normally gives rise to only blood cells; mesenchymal stem cells (blood marrow stromal cells) normally give rise to specific connective tissue cell types; hematopoietic stem cells normally give rise to all types of blood cells; neural stem cells in the brain normally give rise to nerve cells/neurons, astrocytes, oligodendrocytes; epithelial stem cells from the digestive tract lining normally give rise to digestive-specific cell types; skin stem cells retrieved from the basal layer of the epidermis normally give rise to keratinocytes, while follicular stem cells retrieved from the base of the hair follicle of the epidermis normally give rise to hair follicle cells and epidermal cells.

The present invention provides stem cells that are able to differentiate into non-derived tissue (e.g., a cell type that differs from the tissue from which they were derived). This ability is termed plasticity. Thus, stem cells of the present invention are able function by (a) generating a line of genetically identical cells (e.g., same cells or clones) and (b) differentiating into a number of specialized cell types.

Stem cells of the present invention are identified by: (1) labeling cells in a living tissue with molecular markers to determine the specialized cell type; (2) removing the cells from a living tissue (i.e., donor, subject or patient), culturing cells followed by manipulation in vitro (e.g., introduction of one or more nucleic acids sequences or addition of one or more differentiation-inducing substituents) to determine the range of specialized cell types that the cells may become; and (3) removing cells from a living tissue (i.e., donor, subject or patient of the same or different genetic background), labeling cells after they are cultured and then transplanting them back into a host (e.g., donor, different subject or patient of the same or different genetic background) to determine whether the cells repopulate their tissue of origin. Infection of stem cells with a virus that provides a unique identifier to each new stem cell progeny is used to demonstrate that stem cell clones will repopulate one or more injured tissues in a patient in need (e.g., donor or host).

As described herein, the present invention provides methods for developing, recruiting, and retrieving pluripotent stem cells from a body cavity space of an adult mammalian host. In accordance with the present invention, cells retrieved from the body cavity space of the adult mammalian host are able to (a) create a clonal population of stem cells; (b) proliferate both in vitro or in vivo (e.g., in situ), to generate large numbers of stem cell progeny; (c) differentiate into one or more specialized cell-type upon manipulation in vitro (e.g., demonstrate plasticity) or in vivo. Methods for proliferation, manipulation, and differentiation of the stem cells retrieved from a body cavity space of a mammalian host are also provided. As used herein, proliferation, manipulation, and differentiation of the stem cells retrieved from the body cavity space of an adult mammalian host are generally performed in suspension or on a substrate to which the cells adhere (e.g., monolayer, three-dimensional network). A substrate or second cell may be used, one that has one or more adherent molecules, chemicals or biologic substances on its surface (e.g., crosslinked or noncrosslinked amino acids, nucleic acids, polymers, polysaccharides, etc). Alternatively, proliferation, manipulation and differentiation of stem cells may be performed within the host prior to or without retrieval. As used herein, biologic substances are organic compounds that may influence a cell, tissue, or organ.

Proliferation and differentiation of stem cells of the present invention are induced, under appropriate conditions, either ex vivo or in vivo as follows: (a) proliferation and differentiation in vitro followed by incorporation into a host; (b) proliferation in vitro followed by incorporation into a host followed by further proliferation and/or differentiation in vivo; (c) proliferation and differentiation after incorporation into a host (i.e., in vivo). As used herein, proliferation and/or differentiation in vivo (e.g., in a host) may occur in combination with a surgical procedure, injection procedure, a non-surgical approach (e.g., one that coaxes stem cells proliferation using pharmaceutical manipulation), or a combination of these approaches. Proliferation and differentiation are under controlled stimulation when available. In general, conditions for in vitro cell culturing are near physiologic conditions.

When culturing stem cells, culture medium is generally one used under standard conditions appropriate for stem cells or for cell specialization. Supplementation may occur with a growth-promoting signal or substituent, as known to one of ordinary skill in the art. As used herein, the terms “growth-promoting signal,” “growth-promoting substituent,” “proliferation-inducing signal,” “proliferation-inducing substituent,” or “growth factor” generally refer to a compound (e.g., protein, peptide or other molecule) or stimulus having a growth, proliferative, and/or trophic effect on a cell. As used herein, “differentiation-inducing signal,” “differentiation-inducing substituent,” generally refer to a compound or stimulus that induces cell differentiation or specialization. Such compounds may be referred to generally as “signals” or “substituents.” Those of ordinary skill in the art will recognize that one or more substituents may be used in combination and combinations that promote growth, proliferation, and cell-type specific differentiation are known in the art, thus inducing growth, proliferation, and differentiation does not require undue experimentation. Examples of signals or substituents include cytokines, growth-promoting pharmaceutical agents, growth factors such as epidermal growth factor (EGF), amphiregulin, fibroblast growth factor (FGF, acidic or basic), nerve growth factor (NGF), platelet-derived growth factor (PDGF), thyrotropin releasing hormone (TRH), transforming growth factor (TGF), and insulin-like growth factor (IGF), as examples. Such substituents are usually added to the culture medium at concentrations ranging between about 1 fg/ml to 1 mg/ml. To optimize culture conditions, simple titrations can be performed easily to determine optimal substituent concentrations. Additional signals include mechanical and/or electrical signals (e.g., cell-cell contact, adhesion, movement, electrical stimulation, physical pressure, distortion, etc.).

Body Cavity Space

The present invention uses the introduction or implantation of a foreign object in a body cavity space of a mammal (e.g., donor or host) to induce the formation, accumulation and recruitment of stem cells into the body cavity space. Examples include the peritoneal cavity, subcutaneous space, pleural space, lung and/or brain space. These regions are generally larger (biologically), often with significant circumferential area and generally afford easy access, especially as compared with protected and space-limited organs, blood vessels, or bone marrow. As used herein, body cavity space may also be referred to as “cavity,” “peritoneal cavity,” “subcutaneous cavity,” “lung cavity,” “pleural cavity,” “brain space,” and “cavity space.”

As used herein, “foreign object” includes any object (solid, liquid or gel) that is introduced into and induce stem cells to form in the body cavity space, wherein formation includes the migration, recruitment and accumulation of stem cells in the body cavity space. Examples include a degradable implant, non-degradable implant, inflammatory agent (e.g., vaccine adjuvants, dead bacterial fragments), fibrotic agent, pharmaceutical composition, injectable substance (liquid, gel, or solid), biocompatible composition (protein, nucleic acid, polymer, dye, plastic, synthetic or natural drug, chemical isotope, metal composite, etc.), medical instillation solution (e.g., saline, dextran, water) as well as compositions or materials used in a surgical procedure. The biomaterial may be of a single composition or a polymer or composite blend of many materials either in a layer or in a mixture and may include a substance thought to promote biologic growth. In one embodiment of the present invention, the implant of the invention is a biocompatible support making possible the biological anchoring of cells. Alternatively, stem cell initiation is promoted by entry of a foreign object substance such as a medical instillation (e.g., water, dextran, saline) into the body cavity space.

Access to the body cavity space is by surgical approach, injection, perfusion or the like. Surgical techniques include trans-abdominal wall incision, laparoscopic surgical technique (one or more small incisions or using microsurgical instruments to access the cavity), endoscopy (access with a flexible endoscope), etc. Injection is generally with a needle and syringe. One skilled in the art is acquainted with methods and techniques that allow body cavity space access or entry into the body cavity space.

In general, the larger the cavity (e.g., peritoneal cavity, lung cavity, or pleural cavity), the higher the yield of pluripotent stem cells. This is, in part, because larger spaces offer more access for cell migration, larger amounts of fluid can be infused and withdrawn, and, thus, larger amounts of cells may be harvested from the space. When needed, several rounds of infusion and withdrawal may be performed to continuously retrieve stem cells from the body cavity space. In one embodiment, gathering cells from the cavity may be as simple as loading the peritoneum with fluid and draining the fluid out. As such, the present invention has found that the fluid will contain stem cells only after a foreign object is introduced into the cavity. For example, a process similar to that of peritoneal dialysis may be performed in which a catheter is used to fill the abdomen with up to 3 liters of a solution (e.g., dialysis solution) and from the walls of the abdominal cavity (i.e., the peritoneum) the fluid along with other particulates from within the cavity are returned (dialyzed) as a solution when drained from the body.

Stem Cell Retrieval

Stem cells of the present invention are generally retrieved from the cavity of a donor that is a mammal. Any animal with a body cavity space and the ability to generate pluripotent cells may be used as the stem cell donor, including insects, fish, reptiles, birds, amphibians, and mammals, as examples. In accordance with the present invention, stem cells retrieved from the donor are available for a number of applications, including genetic manipulation, diagnostics, phenotyping, screening, and/or transplantation into a heterologous, autologous, or xenogeneic host.

Stem cells from the cavity of a donor are generally retrieved by collecting fluid used to wash the body cavity space. They can also be collected from within and around an implanted material, such as one that includes a three-dimensional matrix with pores. As used herein, washing may include lavage, dialysis, medical instillation and other methods of washing, perfusion, disruption or dissociation in order to collect cells from the body cavity space. Cells may also be collected by dissociation (before or after washing) from any extracellular matrix tissue or implant in the cavity and generally under sterile procedures. Where dissociation and/or disruption is required, routine methods well known in the art are implemented, such as treatment with enzymes (trypsin, collagenase, as examples) or by physical methods (e.g., using a blunt instrument). Fluid used for washing/dissociation may be a regular or modified pH balanced (physiologic) solution such as water, saline, medical instillation solution, dextran, or tissue culture medium (e.g., HEH, DMEM, RPMI, F-12, as examples, used alone or in combination). In one embodiment, cells may be centrifuged at low speed (e.g., 200 to 2000 rpm) and then resuspended in fresh culture medium prior to culturing which may be on a fixed substrate or in suspension. Alternatively, cells are collected with fluid. Fluid used for washing/dissociation may also contain one or more signals, substituents and/or supplements, such as those required for growth, cellular metabolism (e.g., growth factors, amino acids, vitamins, minerals, and/or proteins, as examples) and those that prevent infection or contamination by yeast, bacteria, fungi, etc. (e.g., antimicrobial, antibiotic, antifungal, antiviral). Washing and dissociation fluid as well as fresh culture medium are used with or without serum (e.g., from bovine, equine, chicken, as examples). Routine culture medium used for cell harvesting, inducing growth, differentiation, dedifferentiation, cell expansion, immortalization, specialization and for transplantation are well known in the art. No undue experimentation is required to obtain an appropriate fluid for washing. In addition, methods of optimizing cell collection are routine for one of ordinary skill in the art. Where collected cells are transferred for growth, differentiation, or other manipulations, the appropriate medium is replaced or perfused, either continuously or periodically as needed. Using standard culture techniques, the retrieved/collected cells may be further enriched by any desired amount (e.g., for cell expansion). Different known methods may be used to achieve this enrichment, (e.g., negative selection method or positive selection method). Via this or other procedures known in the art, stem cells and progenitor cells may be concentrated to any degree desired. An alternative means includes using a packing cell line infected with a retrovirus, or a supernatant obtained from such a packaging cell line culture, is added to the stem cells retrieved in accordance with the present invention and when practiced together with an enriched stem cell pool (even in the presence of additional differentiation or proliferation-inducing substituents) provides a very effective means for obtaining stem cell infection in vitro.

Application of stem cells upon retrieval may include: induction of proliferation, cell expansion, differentiation into one or more specialized cell types, genetic modification, short- or long-term storage (e.g., cryopreservation or any method known in the art), screening, diagnostic probing, phenotyping, and therapeutic interventions such as for transplantation, chemotherapy, disease treatment, disease prevention, and cell, organ or tissue replacement or enhancement, as examples.

Adding one or more recruitment-inducing substituents into the cavity of the donor may increase the actual number of stem cells retrieved from the cavity. As used herein, “recruitment-inducing substituents” refer to substituents that stimulate cell recruitment and/or proliferation, examples of which include anti-mitotic agents, anti-differentiating substituents, or proliferation-inducing signals. This ability to enhance the recruitment and proliferation of stem cells retrieved from a subject or donor reduces the time between retrieval and therapeutic intervention and improves the efficiency of the present invention and is one form of cell enrichment.

Proliferation

For inducing proliferation, the culture medium may be generally supplemented with at least one proliferation-inducing substituent or substance (i.e., a chemical or biologic factor, generally trophic, that includes cell division, such as molecules that binds to a receptor on the surface of the cell and exert trophic or growth-inducing effects). Examples of proliferation-inducing growth factors include EGF, amphiregulin, FGFs, TGFs, as examples, used alone or in combination. Additional substituents may be added to the culture medium, especially those that are lineage specific substituents such as vitamins, NGF, PDGF, TRH, TGF, BMP, GM-CSF, or IGF, as examples.

Proliferation of cells of the present invention may occur prior to or after collection from the body cavity space, where similar proliferation-inducing signals may be used. Proliferation of the cells retrieved from the cavity may begin as early as a few hours or may take several days. Generally, cells retrieved from the body cavity space, when placed on a substrate, become adherent within a few hours. After proliferation, new unspecialized cells will appear in culture. These proliferative cells are generally clonal (i.e., are progeny of a single stem cell). In the continued presence of a proliferation-inducing signal, the number of total cells in culture will increase. In addition, the original cells retrieved from the cavity may increase in size. Proliferating cells will continue to proliferate in suspension if continually offered the appropriate culture medium as described above. The proliferating cells in culture may also be passaged and proliferation reinitiated. Importantly, passaging and reinitiating proliferation may be continuously repeated (e.g., weekly) resulting in a logarithmic increase in the number of cells after each passage. Following proliferation and/or passaging, the stem cells may be genetically modified in vitro using techniques as described below.

Differentiation

Differentiation of cells of the present invention may be induced by any method that activates the cascade of biological events leading to growth, including such things as liberating inositol triphosphate (ITP), intracellular Ca²⁺, liberation of diacyl glycerol and/or the activation of protein kinase C (PKC) and other cellular kinases, as examples. Differentiation is controlled by external signals, such as chemical secretions by other cells, physical contact with neighboring cells, and certain other molecules in the environment (e.g., molecular substituents). Other examples of methods that induce differentiation include treatment with phorbol esters, differentiation-inducing growth factors and other chemical signals, alone, in a temporal sequence or in combination with other signals. In addition, plating the cells on a fixed substrate (e.g., flask, culture plate, or coverslip that may also be coated with an ionically charged surface such as poly-L-lysine and poly-L-omithine, as examples) may also induce differentiation. Other substrates that may induce differentiation include those that resemble the extracellular matrix such as collagen, fibronectin, laminin, as examples and may be used alone or in combination. Differentiation may also be induced in a suspension in the presence of a proliferation-inducing substituent.

Differentiation of cells of the present invention occurs in as little as two hours or as long as at least a week, depending on the chosen lineage. After addition of the appropriate differentiation-inducing agent(s) to cells retrieved by methods of the present invention, many of the cells will differentiate. Differentiation and detection of a specific cell-type or lineage may be determined by morphology, immunocytochemistry and/or immunohistochemical methods or by expression of cell-type specific RNA or DNA. For example, cell-type specific antibodies, expression of specific genes, or specific histochemical assays may be used to distinguish cellular characteristics or phenotypic properties of the specialized cells. Those skilled in the art will be able to recognize the methodologies that best characterize a specific cell type. Cells may specialize into one of any cell type depending on the substituent (or temporal sequence thereof) that is added to the cell culture medium. Examples of cell types include neural (e.g., glia, dendrites, etc.), non-neural (astocytes, oligodendrocytes), epithelial, hematopoietic, hepatic, cardiac, endothelial, muscular (smooth and skeletal), epidermal, osteoblastic, osteoclastic, chondrocytic, stromal, adipocytic, as examples.

Genetic Modification and Manipulation

Although the stem cells retrieved from the peritoneum or other such cavity spaces are non-transformed cells, they possess features of a continuous cell line. In the unspecialized state, in the presence of a proliferation-inducing substituent, cells continuously divide and are, therefore, excellent targets for genetic modification. The term “genetic modification” or “genetic manipulation,” as used herein, refers to the stable or transient alteration of the genotype of a cell retrieved from the cavity of an animal by intentional introduction of exogenous nucleic acid. The nucleic acid may be synthetic or naturally derived, and may contain genes, portions of genes, or other useful nucleic acid sequences.

Exogenous nucleic acid may be introduced to a stem cell of the present invention by viral vectors (retrovirus, modified herpes viral, herpes-viral, adenovirus, adeno-associated virus, cytomegalovirus as examples) or mammalian cell-specific promoter or transfection (lipofection, calcium phosphate transfection, DEAE-dextran, electroporation, as examples) that direct the expression of one or more genes encoding a desired protein and may be used to promote differentiation. As used herein, the protein may be any protein or protein combination of interest and may be linked to a selectable marker for detection. In addition, the vectors may include a drug selection marker. (See Maniatis et al., 1982, in Molecular Cloning: A Laboratory Manual, Cold Spring Harbor Laboratory, N.Y. for examples of techniques known in the art.)

An alternative approach is the intentional immortalization of the stem cell by introducing an oncogene that alters the genetic make-up of the cell thereby inducing the cell to proliferate indefinitely. In addition, stem cells, especially those induced to differentiate can be genetically modified to cease cell death by administering Bcl-2 or by genetically modifying the cells with the bcl-2 gene, whose product is known to prevent programmed cell death (apoptosis).

The genetically modified cells of the present invention possess the added advantage of having the capacity to fully specialize to become cell-type specific cells. Specialization is reproducible and may be operable by using any of a number of well-known differentiation protocols. In one embodiment, the genetically modified stem cells retrieved from the cavity are implanted for cell/gene therapy into one or more heterologous and/or autologous hosts in need of one or more biologically active molecules produced by the genetically modified cells. Transplantation techniques are detailed below. Alternatively, the genetically modified stem cells may be subjected to one or more differentiation protocols prior to implantation. Once the cells have specialized, they may be isolated and implanted into one or more hosts in need of the protein or biologically active molecule that is expressed by the genetically modified cell.

Transplantation, Rehabilitation and Therapeutic Uses of Stem Cells

In one embodiment of the present invention, transplantation with one or more stem cells and/or compositions of the present invention is performed in order to treat or prevent one or more disorders, diseases, or degenerative conditions, to repair or replace a damaged, poorly functioning or malfunctioning tissue/organ, or to enhance cell, tissue, or organ function. Such organ or tissue injuries (also referred to as affected areas) may be from mechanical, chemical, molecular, or electrolytic insults, changes or abnormalities. Transplantation with compositions of the present invention may be accompanied by immunosuppression as deemed necessary by one of ordinary skill in the art. In some cases, stem cells with genetic modification that includes gene replacement or gene knockout using homologous recombination may be employed. For example, techniques for ablation of major histocompatibility complex (MHC) genes are well known in the art (Zheng et al., 1991. PNAS, 88:8067-8071). Stem cells lacking MHC expression allows the use of cells of the present invention across allogeneic and xenogeneic histocompatibility barriers without the need to immunosuppress. Additionally, stem cells for transplantation may be retrieved from the cavity of a transgenic animal with altered or deleted MHC antigens.

With transplantation, stem cells and compositions of the present invention are delivered throughout the affected neural area or to one or more specific sites as deemed appropriate to one of ordinary skill in the art. The cells are administered using any method known to maintain the integrity of organ or tissue. In one embodiment of the present invention, transplanted stem cells or specific components of the cells have been genetically modified to include one or more tracers (e.g., dyes or markers such as rhodamine- or fluorescein-labeled microspheres, or fast blue, bisbenzanide, or retrovirally introduced histochemical markers, or isotopic compounds, or light-modifiable chemicals or proteins such as green fluorescence protein, as examples). Here, stem cells can be used as diagnostic markers, for probing, for visualization of tissue or organ remodeling changes, for markers of response to one or more stimuli (e.g., mechanical or chemical, such as electric fields, proteins, chemicals, drugs, etc.) or other therapeutic purposes.

Stem cells of the present invention are also used for bioengineering purposes. In one embodiment, stem cells are recruited in a three-dimensional matrix (e.g., scaffold) that represent the tissue of interest. Upon recruitment, stem cells may or may not be induced to differentiate. Stem cells may remain in the scaffold, where they are genetically modified or induced to differentiate or may be removed from the scaffold where they undergo further manipulation prior use. Importantly, stem cells may be recruited and induced to differentiate in vivo at the site of interest in order to create a new organ and/or tissue. Alternatively, the scaffold is retrieved and implanted elsewhere or donated to a recipient.

Screening, Diagnostics, Testing, and Probing with Stem Cells

Stem cells retrieved from the cavity of the present invention, either those induced to differentiate or not, will be essential for screening of toxins and of potential therapeutic compositions and pharmaceutical preparations. In vitro, compositions are applied to cells at differing dosages, varying times during proliferation and or differentiation and cell response is likewise monitored over time as is well known in the art. Morphologic (physical), genetic, secretory, conductivity (e.g., ion channels or nerve conduction) and other such responses are analyzed by one or more methods well known in the art (e.g., Western blot, Southern blot, Northern blot gene screening, immunohistochemistry, protein, receptor and enzyme assays, enzyme-linked immunosorbant assays (ELISA), electrophoresis analysis, HPLC, radioimmune assays, electrophysiologic measures, as general examples). Similarly, cell type-specific or proliferating stem cells of the present invention may be grown on a feeder layer (acting as a substrate) or in a three-dimensional network. Thus, stem cells, prior to screening, may have already undergone differentiation.

Similar to in vitro screenings and testings, transplanted stem cells of the present invention (with or without the induction to differentiate) in the absence and presence of one or more specific compositions or preparations are observed for their efficacy and safety (e.g., host survival, pharmacologic, biochemical and immunologic effects, etc.). In addition, the stem cells or type-specific cells of the present invention are used to measure the effect of an implant or another transplant on cells or a host.

The term “potential therapeutic compositions” or “pharmaceutical preparations” refer to any agent, such as a chemical, polymer, radioactive substance, virus, protein, peptide, amino acid, lipid, carbohydrate, nucleic acid, nucleotide, drug, pro-drug, implant, and device, as examples. With the present invention, cells that have already been induced to specialize prior to the screening are also screened.

Screening with stem cells of the present invention (either in vitro or in vivo and with or without further induction to differentiate) provides an economic way to test and monitor industrial or biologic chemicals and compounds. In one embodiment, cells are use for rapid identification of substances (e.g., via high throughput screening methods) involved in the proliferation, differentiation and survival of in vitro or host cells (including an organ or tissue). Furthermore, cDNA libraries may be constructed from stem cell or lineage-specific cells of the present invention using techniques known in the art. As such, nucleic acids or their factors involved in cell regulation, dysfunction, repair, remodeling, etc. are analyzed and industrial compositions and/or pharmaceutical preparations are designed to promote positive cell features and counteract negative ones. Diagnostic probes are also developed, especially those that identify one or more genetic disorders or dysfunction. In addition, cells of the present invention are investigated for their ability to secrete or produce potential therapeutic or industrial compositions.

The following examples are presented in order to more fully illustrate the various embodiments of the invention. They should in no way be construed, however, as limiting the scope of the invention, as defined by the appended claims.

### Example of Accumulating and Inducing the Formation of Pluripotent Cells

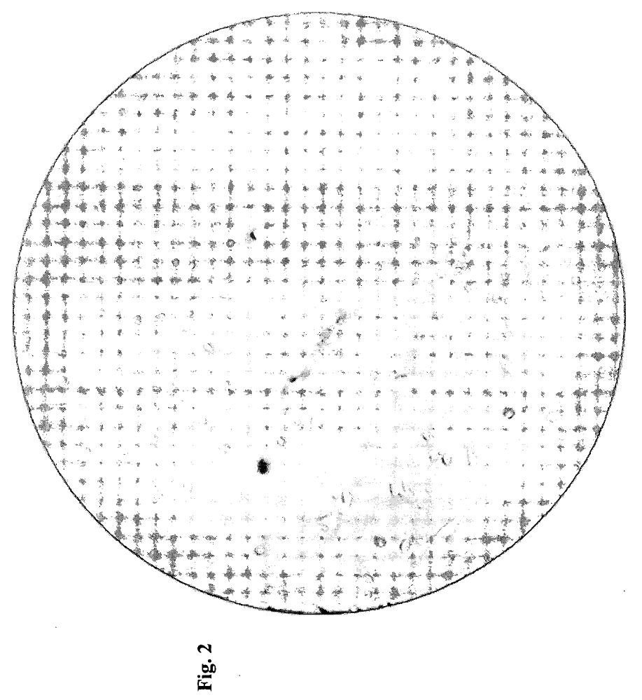

For the present invention, an inflammatory response occurs with the recruitment or retrieval of stem cells in the body cavity space of a mammal. The inflammatory response is generally induced by introducing (surgically or otherwise) a foreign object into the body cavity space. In one embodiment, an implant is surgically placed into the peritoneal cavity of a mouse, wherein the implant includes Polyethylene Terephthalate (PET) disks, and results are compared to those from mice undergoing sham surgery. The induction of pluripotent (e.g., stem) cells in the cavity occurs as early as at least about two hours after the introduction of the implant into the peritoneal space (data not shown). The implant will continue to induce the formation of pluripotent cells for at least about 14 days and longer (data not shown). Cells residing and recruited to the cavity were collected by lavage of the cavity. Collected cells were placed in culture and allowed to adhere to a substrate (e.g., culture plate). In culture, adherent cells were found to expand at an exponential rate. Initially, when viewing adherent cells, several different morphologic features were observed as illustrated in FIG. 2. After days, weeks and/or months of cell growth in culture, stem cells are induced to differentiate into different cell types, including osteoblast, smooth muscle, fibroblast, neurons, and dendritic cells as will be discussed below.

As shown, an inflammatory response as well as a foreign object are necessary for the recruitment and retrieval of stem cell from the peritoneal cavity (e.g., retrieval from lavage fluid). As such, a foreign object is required for stem cells to appear in the lavage fluid and to proliferate in culture (data not shown).

### Example of Stem Cell Retrieval

In one embodiment, cells were recruited by implantation of 1.2 cm diameter PET disks into the peritoneum of Swiss Webster mice by midline incision. Two PET disks—one disk on either side of the peritoneal cavity—were implanted after which the wound was closed with steel wound clips. After introduction of the implant for various time periods, mice were sacrificed, and cells within the peritoneum gathered by lavage. Lavage was performed by injection of at least about 4-5 mL of DMEM media into the peritoneum of the mouse and careful withdrawal of the lavage fluid with a transfer pipette after different time points. The withdrawn medium (lavage fluid) was then placed into a tissue culture flask or cell culture plate with at least about 10% fetal bovine serum (FBS) and 1-5% antibiotics (e.g., penicillin and streptomycin) to protect against possible bacteria from dying macrophages or the gut. Cells that adhere were pluripotent stem cells. Most importantly, after a first lavage, approximately 500,000 stem cells per gram of mouse were retrieved. The same procedure may be repeated on the same subject several more time to continue stem cell recovery. For example, a subject undergoing peritoneal dialysis will retrieve stem cells after every dialysis treatment as long as there is a foreign object in the peritoneal cavity that induces an inflammatory response.

Adherent stem cells were then passaged to maintain them in culture with or without inducing proliferation or differentiation. In the presence of normal culture medium, an antibiotic and with minimal serum supplementation, stem cells continued to grow (e.g., divide into a population of progenitor cells) for at least about a year (data not shown).

### Example of Stem Cell Growth

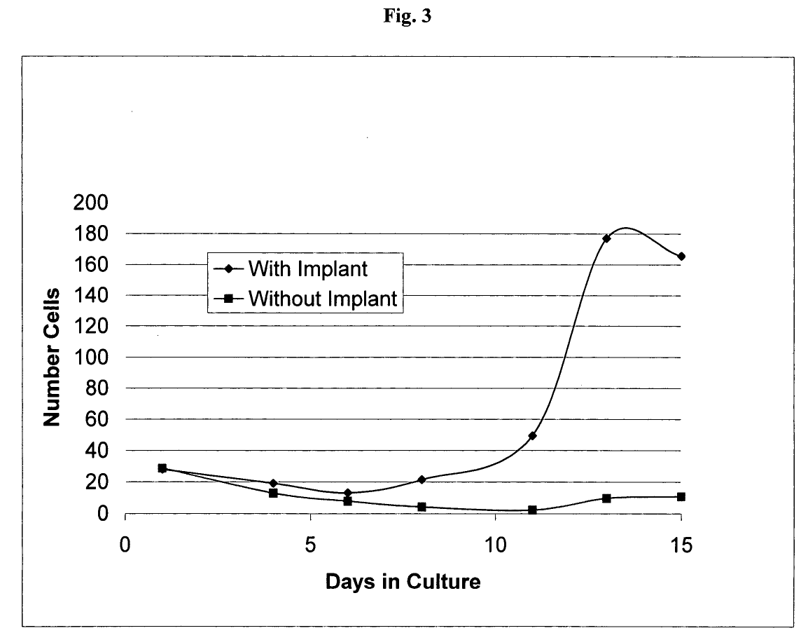

Adherent cells generally with spindle shape morphology were readily recognizable shortly after lavage fluid (used to lavage the peritoneal cavity) was added to a culture surface. Nonadherent cells were removed and fresh media introduced to the cells after sufficient plating time, generally at least about 24 hours. Growth of the adherent spindle shaped cells was monitored by counting them over time in random areas of the culture surface (generally observed under a 40× microscope). FIG. 3 shows that adherent cells grew at an exponential rate (diamonds). On the other hand, cells recovered from the peritoneal cavity of mice in the absence of an implant (i.e., no foreign object introduced into the cavity prior to lavage) and allowed to adhere in the exact same manner as described above did not grow and eventually died out, generally within at least about one week. In general, after the first passage, only stem cells remained in culture. To ensure a pure pluripotent population, alpha-fetoprotein may be used as a marker to further ‘purify’ the population.

### Example of Stem Cell Recruitment

As previously described, induction of an inflammatory or similar such response (e.g., fibrotic or wound healing response) induces stem cells to accumulate and /or migrate into the body cavity space. This occurs in the company of a foreign object, because in the absence of a foreign object, the fluid and its inflammatory components are not able to retrieve stem cells. In one example, cells were gathered from the peritoneum of 16 mice, of which eight animals received an implant and eight did not. Cells were retrieved from the cavity by lavage followed by an additional step to separate lavage fluid from the extracted cells by a brief centrifugation. Lavage fluid was saved following centrifugation and then swapped between the two groups of mice, 4 having received the implant, 4 having not received the implant. The remaining eight animals (4 with implant, 4 without implant) kept their own lavage fluid. Cells from all groups were cultured and spindle shaped cells counted over a number of days.

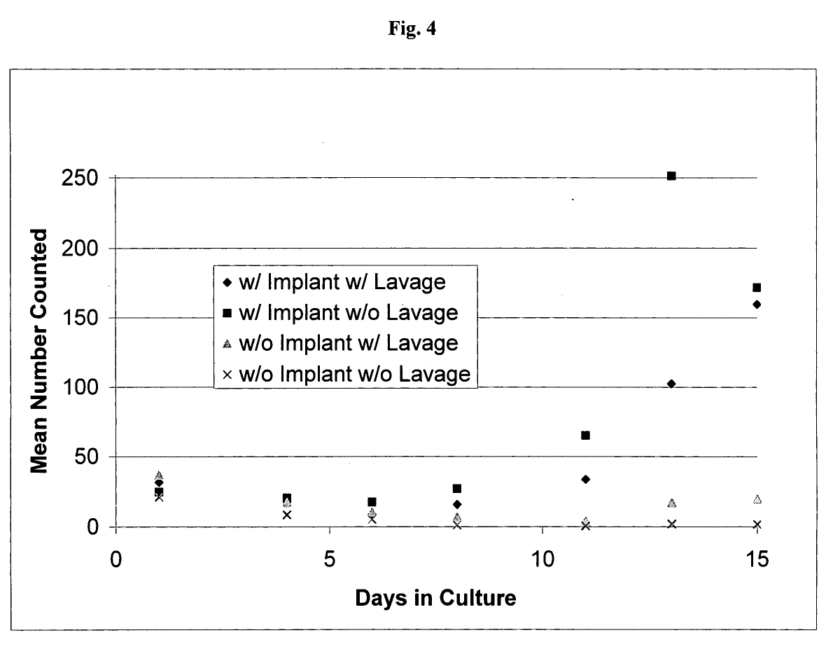

FIG. 4 illustrates the growth curves of these cultured cells. As can be seen from the number of cells counted for more than two weeks, cells from mice that received an implant grew at an exponential rate (diamonds and squares), while cells from mice without implant died (triangles and cross-hatches). Even with the introduction of lavage fluid from mice with implant (containing a multitude of inflammatory proteins and chemokines) to cells retrieved from mice without implant, cells did not grow. Furthermore, FIG. 4 shows that it is the introduction of the foreign object not the lavage fluid itself that induces stem cell formation, infiltration, and retrieval, because cells introduced to an implant grew in the absence (squares) or presence (diamonds) of the lavaged fluid.

### Example of Stem Cell Plasticity

Cells retrieved from the peritoneal cavity of mice following the introduction of a foreign object were cultured for weeks as well as months and remained as stem cells, while some were induced to differentiate. As with normal stem cells, culturing stem cells of the present invention for days or weeks to allow cells to reach confluence can promote differentiation as well as production of an extracellular matrix. Differentiation was also induced by addition of culture-media additives and/or changing culture conditions. Similarly extracellular matrix formation was induced under appropriate conditions. Both behaviors illustrate the plasticity of stem cells of the present invention. Further examples are provided below.

### Example of Specialization into Bone Cells

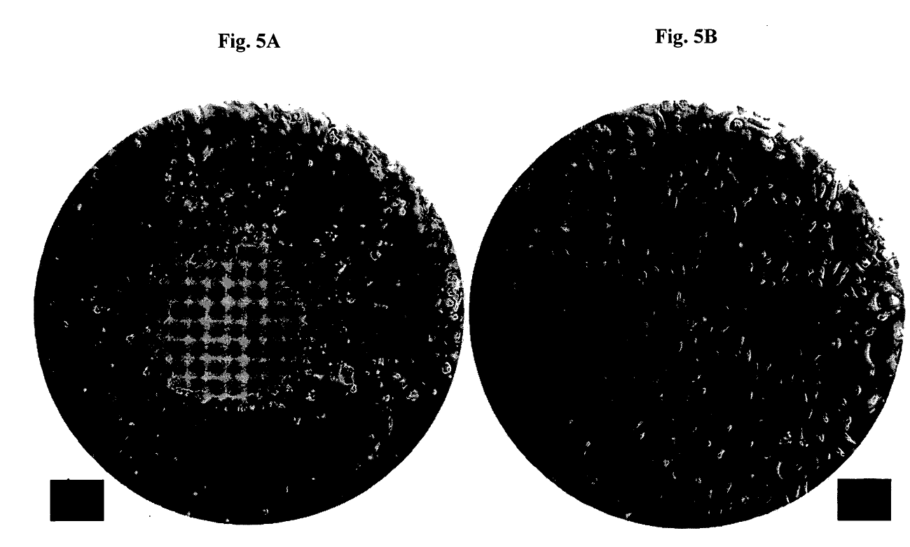

In addition to incorporating culture conditions that induce bone cell formation and are well known in the art, bone cell differentiation by cells of the present invention may occur as follows. In one embodiment, cell aggregation and formation of cultured bone ossicles occurs in cells grown for an extended time and to high density prior to passaging (e.g., near or at confluence) in the presence of DMEM, fetal bovine serum (FBS) and an antibiotic. Cells are also induced to become bone cells as follows. Cells of the present invention are collected from mice with an implant following lavage. Cells are plated in 24-well culture plates using DMEM, 5% FBS and 2% antibiotic. After three hours, fresh media is added to each group (to remove non-adherent cells and lavage fluid). Cells are grown in same media until reaching an approximate density of at least about 5×10⁴ cells/cm². Differentiation-inducing additives (substituents) are then added to cells as follows: (a) DMEM, 5% FBS, and 1000 ng/mL bone morphogenetic protein (BMP)-2; (b) DMEM, 5% FBS, and 300 ng/mL BMP-2; (c) DMEM, 5% FBS, 1% antibiotic and 2.5 ng/mL TGF-β1; or (d) DMEM, 5% FBS, 1% antibiotic (control). Media is replaced with identical additives after at least about one week. After at least about the second week, all groups receive fresh culture media containing DMEM, 5% FBS, 1% PNC, and 4 mmol NaHPO₄ (to enhance extracellular matrix production). After another at least about three weeks with the NaHPO₄—containing media, cells are stained for bone mineralization using a standard Alizarin Red S protocol as is well known in the art, using 2% Alizarin red S mixed in deionized water. FIG. 5A shows an example of the induction into bone cells using BMP-2. Note the calcium rich matrix and surrounding nodules of cells treated with BMP-2, as well as many individual cells on the periphery that also stain red. FIG. 5B shows that stem cells without BMP pretreatment do not show a positive red stain (dark area on image).

### Example of Specialization into Neural Cells

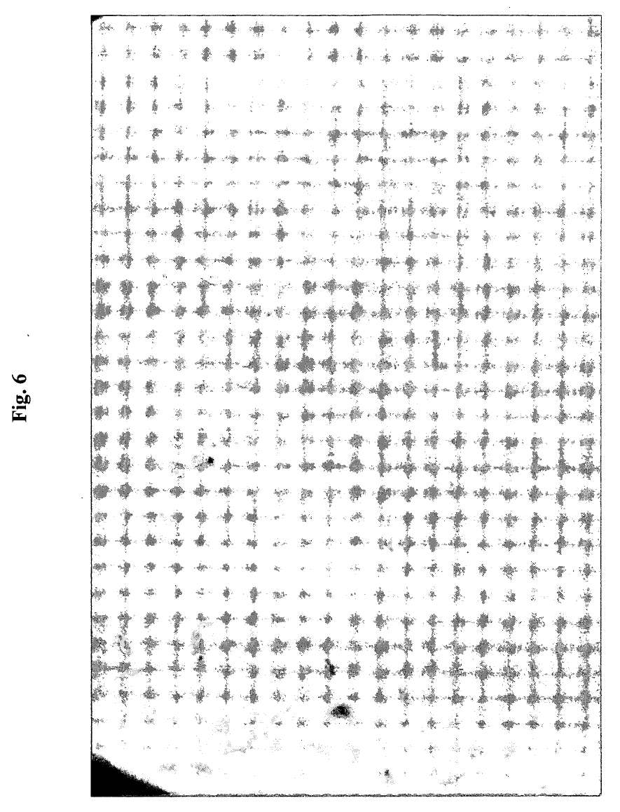

In addition to incorporating culture conditions that induce neural cell formation and are well known in the art, neural cell differentiation by cells of the present invention may occur as follows. As shown in FIG. 6, following incubation with P-mercaptoethanol, cells are capable of differentiation into neural-like cells with a long, branched protrusions from a central cell body. In one embodiment, protrusions may grow to lengths that span over half of the culture plate. Cells of the present invention will form protrusions after a long time in culture (e.g., at least about one month with serum and antibiotics present).

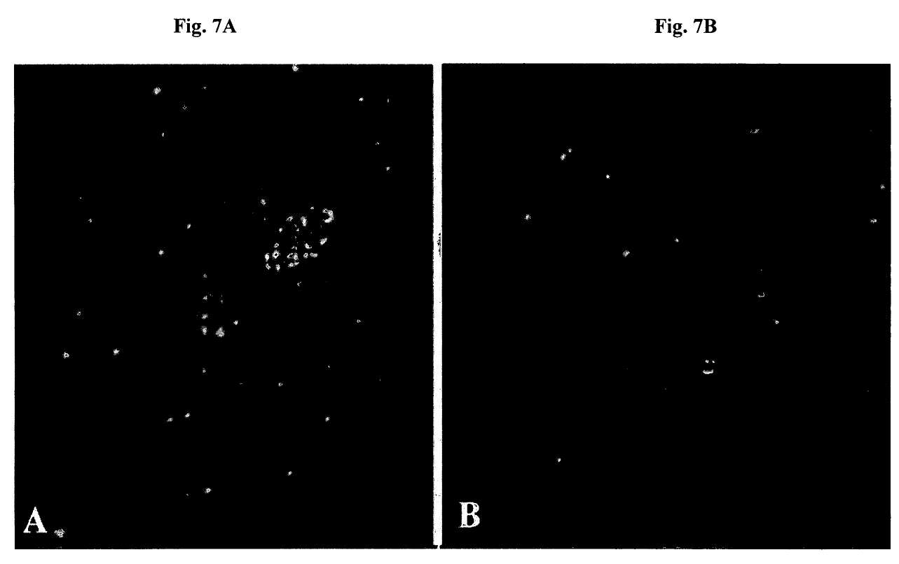

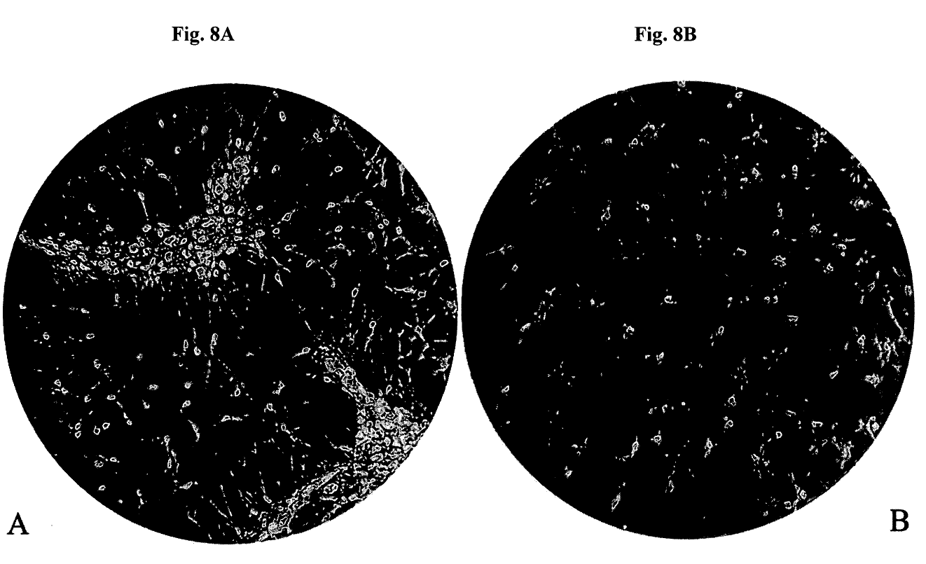

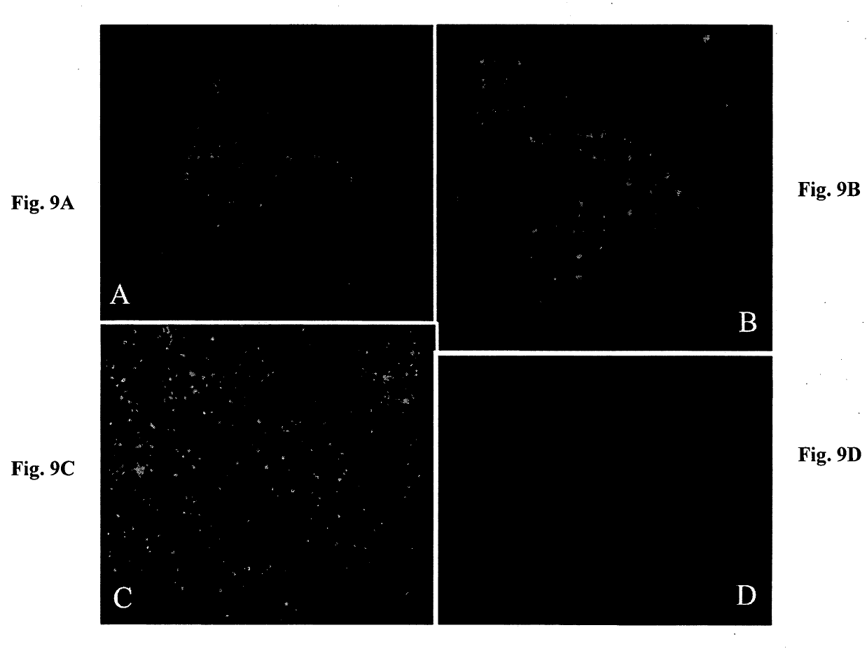

In another embodiment, stem cells retrieved by lavage are induced to become neural cells after culturing with DMEM, 10% FBS as follows. Cells are grown to at least about 50% confluence after which 10 mM β-mercaptoethanol (BME) is added into the medium. At least about twenty-four hours after introduction of BME, morphology of cells will began to change; cell bodies retract and processes elaborate. At least about 48 hours after the addition of BME, cells show a specialized multipolar morphology. FIGS. 7A and 7B are two examples of differentiated neural cells at least about 24 hours after introduction of the differentiation-inducing substituents, a morphology that is not observed before addition of the differentiation-inducing substituents. Cells have been further identified as neural cells by H&E staining (FIGS. 8A and 8B) and immuno-histochemical staining for neuron-specific enolase (NSE) and neurofilament-M (NFM) (see FIGS. 9A, 9B, and 9C). Only neural cells induced to differentiate with neural-specific induction substituents stain positive for both NSE and NFM.

### Example of Specialization into Skeletal Muscle Cells

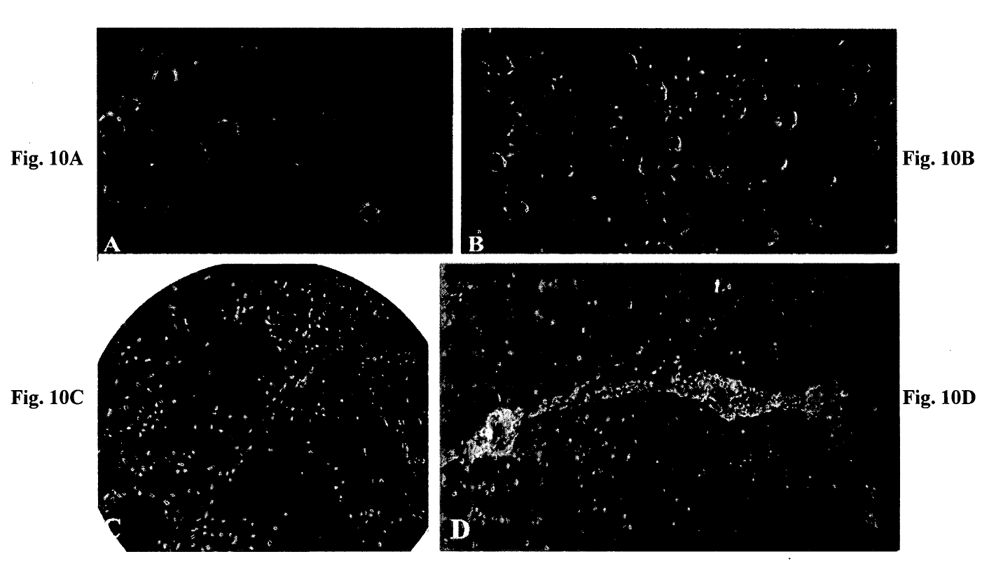

Skeletal muscle cell differentiation by the cells of the present invention may occur by implementing culture conditions that induce skeletal muscle cell formation as well known in the art, and by implementing the following conditions. At least about several weeks after stem cells retrieved from the peritoneal cavity by lavage are introduced to a culture surface, skeletal muscle cell morphology will begin in the presence of DMEM, FBS an antibiotic, and 5-azacytidine. Examples of how these cells look morphologically are shown in FIG. 10. Note the skeletal muscle striations seen clearly in both FIGS. 10A and 10B and after more culturing, myotubes are formed and apparent as in FIGS. 10C and 10D.

### Example of Specialization into Smooth Muscle Cells

Cells of the present invention may be induced to differentiate into smooth muscle cells by implementing culture conditions well known in the art that induce smooth muscle cell formation and by implementing the following conditions. After retrieving cells from the peritoneal cavity and culturing for at least about two weeks, sometimes with two subcultures, in DMEM, FBS and an antibiotic, RNA extracted from the stem cells demonstrate similar morphology and expression of specific proteins that exist predominantly or specifically in proliferative smooth muscle cells. For example, after at least about two weeks in culture, both smooth muscle cells and stem cells of the present invention express collagen type III and smooth muscle actin heavy chain-5′. (Data not shown)

### Example of Specialization into Dendritic Cells

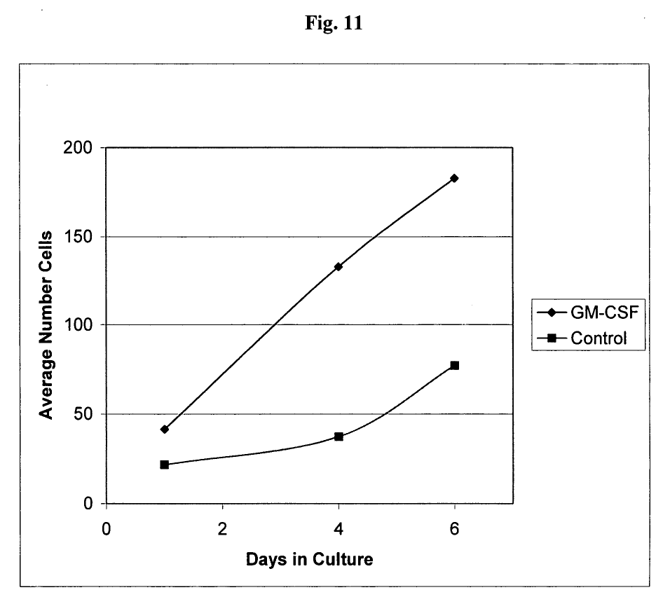

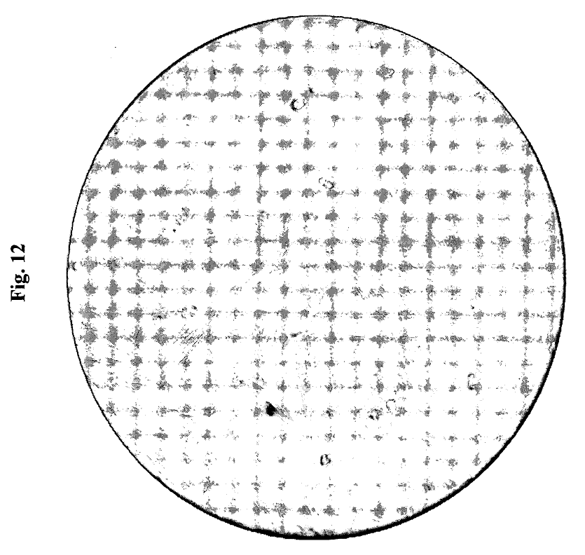

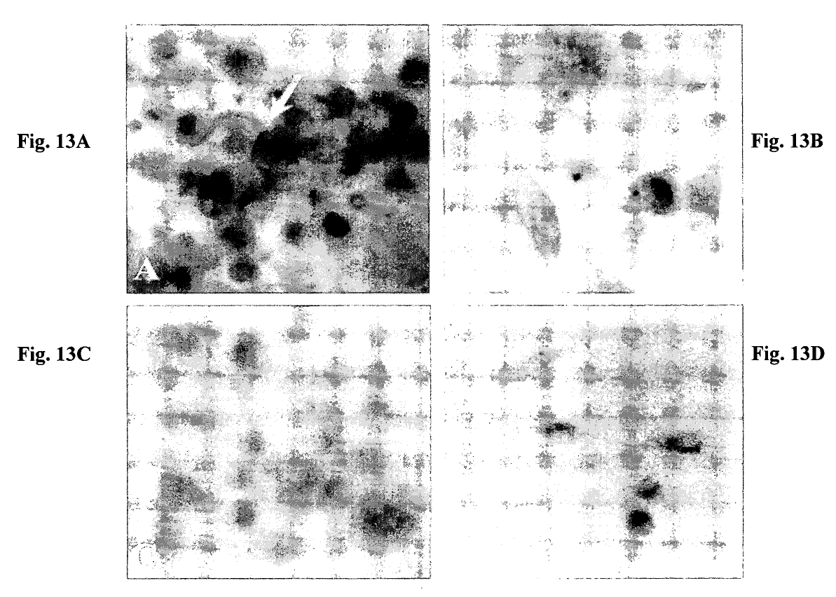

Cells of the present invention may be induced to differentiate into dendritic cells by implementing culture conditions well known in the art that induce dendritic cell formation and by implementing the following conditions. Cells of the present invention retrieved from the peritoneal cavity of a mouse with an implant are gathered by lavage as previously described. Cells are suspended in DMEM, with at least about 10% FBS and 1% antibiotic. Cells are then cultured onto a 24 well plate for at least about 3 hours, after which non-adherent cells are discarded and fresh media (as above) is added to cells. Cells are divided into a control group and one induced to differentiate. In one embodiment, the differentiation-inducing substituents include GM-CSF (20 ng/mL) and IL-4 (20 ng/mL). After addition of such substituents, morphological changes occur, generally uniformly, across the culture plate that included the expansion of each cells (e.g., longer morphology than control) and a higher rate of growth as shown in FIG. 11. FIG. 11 shows the growth profile of control cells (squares) and those receiving the differentiation-inducing substituents of GM-CSF and IL-4 (diamonds) as observed for six days. After at least about one week in culture, fresh media was added. Here control cells received the same media as above (DMEM, FBS and an antibiotic), while the highly proliferating received the same media in addition to GM-CSF (20 ng/mL) and TNF-α (20 ng/mL) to induce dendritic cell maturation (see FIG. 12A for an example of dendritic cells in culture). Cells were then cultured for at least about 24 hours and then fixed in 10% formalin in order to perform immunostaining for the presence of dendritic cell markers (see FIGS. 12B and 12C). As is known in the art, dendritic cells express MHC II, CD86, and CD40, among other markers. In addition, recent studies have shown the dendrites may be selected by their expression of CD11c. Hence cells immunostained with an antibody to CD11c (e.g., antibody bound magnetic beads) are believed to be dendritic cells. FIG. 13A and 13B show that cells induced to differentiate into dendritic cells as described above are also positive for CD11c (as shown using an anti-CD11-FIT-C linked antibody, see arrows). Positive fluorescence is most noticeable around the cell periphery. On the other hand, the CD11c antibody is not detected on the surface of control stem cells (without the differentiation-inducing signal) as shown in FIGS. 13C and 13D.

### Example of Specialization into Adipocytes

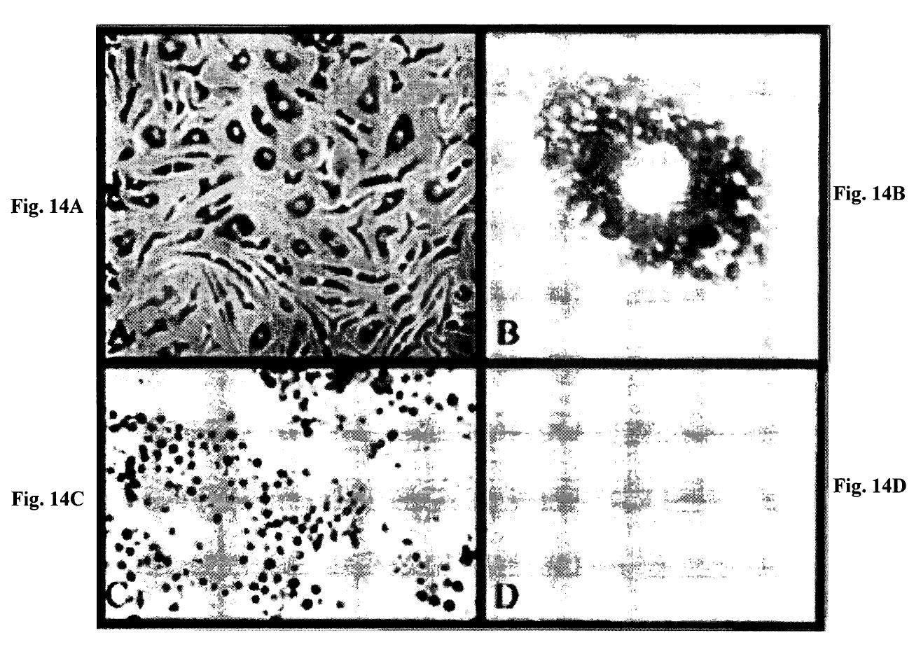

Cells of the present invention may be induced to differentiate into adipocyte and fat cells by implementing culture conditions well known in the art that induce adipocyte and fat cell formation and by implementing the following conditions. Cells retrieved from the peritoneal cavity by lavage were cultured in DMEM with 20% FBS in DMEM and grown to ≧90% confluence. Culture media was then replaced with α-MEM, 10% FBS, 10% rabbit serum, 10% dexamethasone, 5 μg/mL insulin, and 50 μM 5,8,11,14-eicosatetraynoic acid. After at least about two days in culture, cells were cultured in the same media except without dexamethasone. Cells were then maintained for at least one week during which the induction of adipocytes generally is found to begin. The cells are indicative of adipocytes as they are larger, rounder and display very large yellow sacs inside the cell membrane. Cells were identified as adipocytes by staining with Oil Red O (a stain more soluble in lipid reservoirs than the rest of the cell matrix or body) as shown in FIGS. 14A and 14B. Stem cells not induced to differentiate did not stain positive for Oil Red O (FIG. 14D).

### Examples of Several Protocols Used to Induce Differentiation/cell Specialization

Nerve cell specialization protocol:
1. Expand to 70% confluency with 20% FBS
2. Induce at least about 24 hours with 1 mM β-Mercaptoethanol in DMEM, 20% FBS
3. Refresh media with 10 mM β-Mercaptoethanol in DMEM, 20% FBS

An alternative nerve cell specialization protocol:
1. Co-culture with an neuronal, glial, Schwann, or astrocyte cell line
2. Feed with conditioned media obtained from neuronal, glial, Schwann, or astrocyte cell line, or introduce at least one extra cellular matrix protein

Adipocyte cell specialization protocol:
1. Expand to ≧90% confluency with DMEM, 20% FBS
2. Replace media with α-MEM, 10% FBS, 10% rabbit serum, 10-8 dexamethasone, 5 μg/mL insulin, and 50 μM 5,8,11,14-eicosatetraynoic acid
3. After at least two days, feed with same media as in 2 but without dexamethasone
4. Differentiation may begin as early as a few days or take at least about week

Dendritic cell specialization protocol:
1. Culture and grow cells for at least about or up to 9 days with DMEM, serum and GM-CSF combined with IL-4 and/or SCF (stem cell factor)
2. Replace with fresh media every 3-5 days
3. Follow with a dose of GM-CSF and TNF-α for at least 24 hours

Muscle cell specialization protocol (including myoblasts, myotubes, and cardiomyocytes)
1. Grow cells in DMEM with 10-20% FBS (optionally, and 5% horse serum) and do not allow cells to reach at least about >90 confluency
2. Replate cells to achieve at least about 80% confluency
3. Feed with DMEM containing 5-azacytidine or 5-aza-2′-deoxycytidine
4. After 24 hrs. Remove media with 5-azacytidine or 5-aza-2′-deoxycytidine and reefed with same media absent the additive
5. Continue culturing and myotubes generally appear after at least about 7 days

An alternative muscle cell specialization protocol:
1. Co-culture with a myoblast or cardiomyoblast cell line
2. Feed with a myoblast-conditioned medium or with an extracellular matrix protein

Cartilage cell specialization protocol (including chondrocytes and chondroblasts):
1. Culture cells with DMEM, serum and an antibiotic
2. Add media containing FGF, and/or IGF-I, and/or TGF-β1

An alternative cartilage cell specialization protocol:
1. Culture cells with DMEM, serum and an antibiotic
2. After at least a few weeks, introduce cells into any 3-dimensional matrix (e.g., biomaterial-based, polymer-based, or containing hyaluronic acid, proteoglycan, collagen, and/or demineralized bone, as example)
3. Optionally: Introduce a strain gradient to the culture condition (using protocols well known in the art) to enhance extracellular matrix production

Bone cell specialization protocol (including osteoblast and osteocyte):
1. Grow cells in DMEM with serum and an antibiotic until at least about ≧90% confluency
2. Replace with same media containing recombinant BMP-2 (or another BMP)
3. Optionally: Add another dose of recombinant BMP-2 at least about 12-24 hours later
4. Culture and nodules will be recognizable by the naked eye

An alternative bone cell specialization protocol:
1. Culture cells with DMEM, serum and an antibiotic
2. After at least a week, introduce cells into any 3-dimensional matrix (e.g., biomaterial-based, polymer-based, or containing hyaluronic acid, proteoglycan, collagen, and/or demineralized bone, as example)

The present invention provides a means for generating large numbers of unspecialized stem cells as well as multiple types of specialized cells by implanting a foreign object in a body cavity space of a mammal and allowing stem cells to accumulate in the body cavity space. As such, greater that 200-fold the number of stem cells may be retrieved by methods of the present invention, as compared to methods currently used for the retrieval of stem cells (e.g., from the bone marrow). Accumulated stem cells of the present invention (specialized and/or unspecialized) are amenable to in vivo or ex vivo use. Therefore, stem cells of the present invention may accumulate in the host mammal and remain within that host either as stem cells or following induction to a specialized cell type or be retrieved from the host and used for another subject in need thereof (e.g., for bioengineering purposes). Similarly, stem cells of the present invention are amenable to multiple commercial uses such as for cell and product screenings, as a diagnostic tool (e.g., biologic marker), for prophylactic and therapeutic treatment of one or more conditions involving a cellular dysfunction or abnormality, and can be used to create established stem cell lines, cDNA libraries, to produce therapeutic agents, and for genetic engineering. As such, cells, cell lines, and compositions of the present invention may be used for numerous applications, including for transplantation, engraftment, rehabilitation, cell replacement, diagnosis, compound and drug screenings, bioengineering, and the like.

Unlike stem cells retrieved by previously established methods, stem cells of the present invention exhibit true plasticity, because they may be induced to differentiate into bone marrow-derived hematopoietic stem cells, bone marrow-derived mesenchymal stem cells, and neuronal cells of the brain and spinal chord.

In accordance with another aspect of the present invention, a method for inducing the formation of pluripotent cells with differentiation and proliferation capabilities is provided by implanting a foreign object in a body cavity space of a mammal a foreign object and allowing stem cells to accumulate. Further, another aspect is a method of retrieving pluripotent cells with proliferation and differentiation capabilities from a body cavity space of a mammal by washing the body cavity space with physiologic solution following the introduction of a foreign body into the body cavity space. Importantly, the physiologic solution may include additional substituents such as peptides, cytokines, antibiotics, anti- and/or pro-inflammatory agents, anti-oxidants, other nutrients, growth-inducing signals, proliferation-inducing signals, differentiation-inducing signals, and/or differentiation-blocking signals as examples.

In accordance with still another aspect of the present invention, a method of creating an environment for the accumulation of stem cells with differentiation and proliferation capabilities is provided by introducing a foreign object into a body cavity space of an mammal for at least about two hours.

Yet another aspect of the present invention is a method of treating a disease in a host with stem cells retrieved from a body cavity space of a mammal by retrieving stem cells that accumulate in the body cavity space of the mammal following the introduction of a foreign object into the body cavity space followed by introducing the retrieved cells into a patient in need thereof. Stem cells are generally allowed to proliferate (e.g., expand) and may be induced to differentiate before they are introduced into the patient. Stem cells are generally used to treat any disease for which there is a cellular abnormality, including a genetic disease, aging and age-related disorders, an acquired disease, tumor (e.g., cancer), as well as for tissue injury (e.g., damage or inflammation) and repair. In still another aspect, one or more stem cells proliferate, undergo genetic manipulation (e.g., addition of one or more viral, pharmaceutical, or biologic substances, such as proteins, genes, peptides, labels, cell or chemoprotectants, etc.), and/or induced to differentiate.

Another aspect of the present invention is a method of creating a clonal population of stem cells. As such, stem cells may be induced to differentiate, proliferate, and/or introduced into a subject, wherein cells of the desired cell type are typically present in the subject. Further, a pluripotent stem cell is provided in addition to a method for providing to a subject one or more pluripotent stem cells. Still further, a cDNA library, methods of constructing such library, diagnostic probes obtained from such a cDNA libraries are provided. As such, nucleic acids or their factors involved in cell regulation, dysfunction, repair, remodeling, etc. are analyzed and compositions and/or pharmaceutical preparations are designed to promote positive cell features and counteract negative ones.

In accordance with still another aspect of the present invention, a method for immortalizing cells retrieved from a body cavity space of an mammal is provided by introducing a foreign object in a body cavity space, retrieving cells that accumulate in the body cavity space and after culturing, introducing an immortalization gene to the cells under conditions permissive for the uptake of the immortalization gene and allowing cells to self-renew and specialize.

Still another aspect of the present invention is a stem cell (or population thereof) retrieved from a body cavity space of an mammal and immortalized with one or more genes capable of producing a protein, wherein the stem cell is capable of expressing the protein upon induction by agents that stimulate production of the protein.

Yet another aspect of the present invention provides a composition that includes a stem cell retrieved from a body cavity space of a mammal that has been induced to express at least one characteristic of a non-cavity derived cell and a pharmaceutically acceptable carrier, and (a) wherein at least one non-endogenous nucleic acid sequence has been introduced into the cell, (b) wherein at least one non-endogenous peptide has been introduced into the cell, and/or (c) wherein at least one monoclonal antibody has been introduced into the cell. The characteristic includes those defined as neuronal, astroglial, dendritic, hematopoietic, hepatic, cardiac, skeletal, epithelial, adipocytic, alveolar, ocular, endothelial, osteoblastic, chondrocytic, epidermal, pancreatic, renal, tenocytic, and reproductive, as examples. These stem cells may possess cell functions (e.g. the function of liver cells, kidney cells), physical structure (bone or fat cells) or produce essential proteins or biological substances (eg. insulin, growth factors) for the treatment of diseases such as diabetes, liver or kidney failure, plastic surgery, and the like.

In yet another aspect of the present invention, a stem cell retrieved from a body cavity space of a mammal operable for identifying substances involved in the growth of in vitro or host cells is provided, in addition to a method of testing such substance. The substances may include approved and investigational pharmaceutical products, agricultural agents, food products, chemical products used for industrial purposes, and known or suspected toxins, as examples.

The present invention also provides a composition and a method for generating cells to be used for cell-based therapies, wherein cells retrieved from a body cavity space of an mammal are directed to differentiate into one or more specialized cell type and serve as a renewable source of replacement cells introduced into a patient to treat a human disease (e.g., transplant into a damaged or disease tissue or organ, repopulate an organ or tissue, integrate into surrounding tissue after transplantation). Cells may be introduced and allowed to integrate as a primary or secondary cell source as needed.

Additional objects, advantages and novel features of the invention as set forth in the description that follows, will be apparent to one skilled in the art after reading the foregoing detailed description or may be learned by practice of the invention. The objects and advantages of the invention may be realized and attained by means of the instruments and combinations particularly pointed out here.

1. A method of collecting pluripotent stem cells comprising the steps of:
introducing a foreign object into a body cavity space of a mammal;
washing the body cavity space at least once with a physiologic solution; and
collecting the physiologic solution to retrieve pluripotent stem cells.

2. The method of claim 1, wherein the body cavity space is one or more of the group consisting of a peritoneal cavity, subcutaneous space, pleural space, lung space, and brain space.

3. The method of claim 1, wherein the physiologic solution is selected from the group consisting of culture media, dextran, saline, serum, antibiotic, medical instillation solution, and combinations thereof.

4. The method of claim 1, wherein the step of washing is performed at least about two hours after the introduction of the foreign object into the body cavity space and is optionally repeated at least about every two hours.

5. The method of claim 1, wherein the step of washing is chosen from the group consisting of lavage, dialysis, and medical instillation.

6. The method of claim 1 further comprising the step of genetically manipulating the pluripotent stem cells.

7. The method of claim 1, wherein the pluripotent stem cells are reintroduced into a patient in need thereof.

8. The method of claim 1, wherein pluripotent stem cells are induced to differentiate into dendritic cells by incubating in a medium further comprising serum and GM-CSF and optionally replating the dendritic-type cells in a medium comprising serum and TNF-α.

9. The method of claim 1, wherein pluripotent stem cells are induced to differentiate into bone cells by incubating in a medium further comprising serum and a bone-specific growth factor selected from the group consisting of bone morphogenetic protein and TGF-beta₁.

10. The method of claim 1, wherein pluripotent stem cells are induced to differentiate into neuronal cells by incubating in a medium further comprising serum and beta-mercaptoethanol.

11. The method of claim 1, wherein pluripotent stem cells are induced to differentiate into muscle cells by incubating in a medium further comprising serum and an antibiotic and allowing cells to grow for several weeks without reaching full confluency.

12. The method of claim 1, wherein pluripotent stem cells are induced to differentiate into fat cells by incubating in a medium further comprising serum, dexamethasone, insulin, and 5,8,11,14-eicosatetraynoic acid.

13. The method of claim 1, wherein pluripotent stem cells are induced to differentiate into cartilage cells by incubating in a medium further comprising serum and one or more growth factor selected from the group consisting of FGF, IGF-1, and TGF-beta₁.

14. A method of treating a disease in a patient in need thereof comprising the steps of:
collecting pluripotent stem cells from a body cavity space of a subject;
culturing the pluripotent stem cells; and
introducing the pluripotent stem cells into a patient in need thereof.

15. The method of claim 14 further comprising the step of inducing pluripotent stem cells to differentiate in the presence of a differentiation-inducing signal.

16. The method of claim 14, wherein pluripotent stem cells are collected by washing the body cavity space at least about two after introducing a foreign object into the body cavity space.

17. The method of claim 14, wherein the subject is the patient.

18. The method of claim 14 further comprising the step of manipulating the pluripotent stem cells by introducing one or more selected from the group consisting of nucleic acids sequence, amino acid, antibody, differentiation-inducing signal, proliferation-inducing signal, biologic compound, chemical, selectable marker, three-dimensional scaffold, and combinations thereof.

19. The method of claim 14, wherein the pluripotent stem cells are manipulated in vivo.

20. A population of cells obtained by the method of claim 1 comprising:
introducing a foreign object into a body cavity space of a mammal;
washing the body cavity space at least once with a physiologic solution; and
collecting the physiologic solution to retrieve a population of cells.

21. A stem cell retrieved from a body cavity space of a mammal at least about two hours after introducing a foreign object in the body cavity space and able to create a clonal population of stem cells.

22. The stem cell of claim 20, wherein the stem cell is used for one or more applications selected from the group consisting of genetic manipulation, screening, diagnosis, phenotyping, heterologous transplantation, autologous transplantation, engraftment, cell and tissue replacement, cell and tissue enhancement, treatment of cell abnormalities and combinations thereof.

23. A method of forming stem cells comprising the step of:
implanting a three-dimensional object in the body cavity space of a subject, wherein the three-dimensional object acts as a scaffold for the accumulation and growth of stem cells.

24. The method of claim 23, wherein stem cells are manipulated in vivo by selecting from the group consisting of genetic modification, inducing recruitment, inducing differentiation, inducing growth, inducing proliferation, and combinations thereof.

25. The method of claim 17, wherein stem cells are used to treat a condition, wherein the condition is selected from the group consisting of tumor, disease, cancer, tissue injury, cell death.

26. A population of cells retrieved from a body cavity space of a subject induced to express at least one characteristic of a non-body cavity space-derived cell and suitable for implantation into a host, wherein the population of cells are retrieved at least about two hours after the introduction of a three-dimensional foreign object into the body cavity space.

27. An isolated stem cell retrieved from a body cavity space of a subject immortalized with one or more genes capable of producing a compound, wherein the isolated stem cell is capable of expressing the compound upon induction by signals that stimulate its production and wherein the isolated stem cell is retrieved at least about two hours after the introduction of a foreign object into the body cavity space.

28. A composition comprising:
a stem cell retrieved from a body cavity space of a subject that has been induced to express at least one characteristic of a non-body cavity space-derived cell, wherein the stem cell is retrieved at least about two hours after the introduction of a foreign object into the body cavity space; and
a pharmaceutically acceptable carrier.

29. The composition of claim 28, wherein the characteristic is selected from the group consisting of neuronal, astroglial, dendritic, hematopoietic, hepatic, cardiac, skeletal, epithelial, adipocytic, alveolar, ocular, endothelial, osteoblastic, chondrocytic, pancreatic, lymphatic, epidermal, renal, tenocytic, and reproductive.

30. A composition comprising:
a stem cell retrieved from a body cavity space of a subject that has been induced to express at least one characteristic of a non-body cavity space-derived cell, wherein at least one non-endogenous nucleic acid sequence has been introduced into the stem cell and, wherein the stem cell is retrieved at least about two hours after the introduction of a foreign object into the body cavity space; and
a pharmaceutically acceptable carrier.

31. A composition comprising:
a stem cell retrieved from a body cavity space of a subject that has been induced to express at least one characteristic of a non-body cavity space-derived cell, wherein at least one non-endogenous amino acid sequence has been introduced into the stem cell and wherein the stem cell is retrieved at least about two hours after the introduction of a foreign object into the body cavity space; and
a pharmaceutically acceptable carrier.

32. A composition comprising:
a stem cell retrieved from a body cavity space of a subject that has been induced to express at least one characteristic of a non-body cavity space-derived cell, wherein at least one monoclonal antibody has been introduced into the stem cell and wherein the stem cell is retrieved at least about two hours after the introduction of a foreign object into the body cavity space; and
a pharmaceutically acceptable carrier.

33. A stem cell derived from a body cavity space of a mammal operable for identifying substances involved in the growth of in vitro or host cells, wherein the stem cell is retrieved at least about two hours after the introduction of a foreign object into the body cavity space.

34. A method of testing the safety of prepared compositions on stem cells retrieved from a body cavity space of a mammal comprising the steps of:
collecting stem cells retrieved from the body cavity space of mammal by washing the body cavity space with a physiologic solution at least about two hours after introducing a foreign object into the body cavity space; and
incubating the stem cells in appropriate media in the presence one or more prepared compositions.

35. The method of claim 34 further comprising the step of examining stem cells at various times after incubation to assess cellular changes.

36. A stem cell line obtained by the method of claim 1 and used to construct a cDNA library.

37. The stem cell line of claim 36, wherein one or more diagnostic probes are obtained from the cDNA library.

38. The stem cell line of claim 36, wherein the stem cells produce one or more therapeutic compositions.

39. A method of collecting pluripotent stem cells comprising the steps of:
introducing a foreign object into a body cavity space of a mammal; and
retrieving pluripotent stem cells from the body cavity space.
